(19) United States
(12) Patent Application Publication
(10) Pub. No.: US 2025/0229466 A1
(43) Pub. Date: Jul. 17, 2025

(54) LAMINATE HAVING IMPROVED TENSILE PROPERTIES

(71) Applicant: Reckitt Benckiser Health Limited, Slough, Berkshire (GB)

(72) Inventors: Miriam Hussain, Hull (GB); Nima ROOHPOUR, Slough Berkshire (GB); Manus SRIRING, Udonthani (TH)

(21) Appl. No.: 18/851,411

(22) PCT Filed: Mar. 31, 2023
(86) PCT No.: PCT/GB2023/050871

§ 371 Date: Sep. 26, 2024

(30) Foreign Application Priority Data
Mar. 31, 2022 (GB) 2204720.3

(51) Int. Cl. B29C 41/22; A61F 6/04; B29C 37/00; B29C 41/00; B29C 41/46; B29K 75/00; B29L 31/00; C08J 7/04; C08K 3/36

(52) U.S. Cl. CPC B29C 41/22; A61F 6/04; B29C 37/0017; B29C 37/0025; B29C 41/003; B29C 41/46; C08J 7/0427; C08K 3/36; B29K 2075/00; B29K 2995/0046; B29K 2995/0097; B29L 2031/7538; C08J 2375/04; C08K 2201/005; C08K 2201/006; C08K 2201/011

(57) ABSTRACT

The present invention relates to a method of preparing an elastomeric laminate article, the method comprising: providing a first aqueous coating composition comprising a first polymer and discrete silicon dioxide nanoparticles, wherein the first polymer is a polyurethane; providing a second aqueous coating composition comprising a second polymer; coating a substrate with the first aqueous coating composition to form a first film on the substrate; coating the first film with the second aqueous coating composition to form a second film on the first film; and removing the substrate. In another aspect, the invention relates to an elastomeric laminate article which comprises a plurality of polymeric films and one or more interfaces, wherein each adjacent pair of films defines one of the interfaces, wherein at least one of the plurality of polymeric films comprises a polyurethane, wherein the laminate comprises silicon dioxide nanoparticles, and wherein at least 60 wt % of the silicon dioxide nanoparticles are present at one or more of the interfaces defined at least in part by a polyurethane-comprising film. In both cases the laminate article may be a condom, and the invention further relates to a condom obtained or obtainable by the inventive method.

The present invention relates to a laminate having improved tensile properties and a method for preparing this. The laminate is an elastomeric article, which may e.g. be a film to be used in contact with the human body (such as in contact with skin), for instance as a barrier during sexual activity or during a medical procedure. In an embodiment, the present invention relates to a condom having improved tensile strength compared to those of the prior art while having comparable softness and elasticity.

The ability of a condom to maintain its integrity throughout sexual intercourse is essential to its role as a contraceptive and in preventing the spread of sexually transmitted infections. A condom must also be highly deformable, while at the same time being thin and flexible enough to allow sensitivity of touch and feel. A number of polymeric materials have been found to be suitable for this purpose. Natural rubber latex (“NRL”) has been used as a condom material for many years, while synthetic polyisoprene (“PI”) condoms have been developed as an alternative for those suffering from latex allergies. Polyurethane (“PU”) condoms are also known. PU condoms can be made to have thinner walls than NRL and PI condoms because of the inherent strength of the PU material, but are typically less elastic or soft. Thin condoms are desirable to some consumers, in part because they may afford less reduction in sensation and pleasure compared with condoms having thicker walls. Polyurethane condoms may also display improved heat transfer properties compared to NRL, resulting in a more pleasurable experience for consumers as sensations are enhanced.

A number of attempts have been made to improve the mechanical properties of the elastomeric films from which condoms are formed. However, it is often difficult to provide an improvement in one mechanical property without adversely affecting another. Reinforcing agents that have been previously used in elastomers include silica, carbon black, carbon nanotubes and graphene. However, due to their rigid nature, the incorporation of these materials also tends to increase the hardness and stiffness of a material (reflected by an increased elastic modulus) and reduces the elongation to break. This is disadvantageous for condom applications, for which high-modulus materials are typically avoided because they may decrease feel or sensitivity.

Similar principles apply to other elastomeric laminate articles, such as gloves, finger cots, dental dams, balloon catheters and so on. Such articles may be made by dipping, for example.

US 2017/0333602 A1 discloses the use of nanocellulose as an additive for an elastomeric material, which may include a polyurethane, for the purpose of providing enhanced tensile strength and toughness while avoiding significantly increasing the elastic modulus or stiffness.

There remains a need for further methods for improving the tensile properties of laminates such as condoms. In particular, there remains a need for further methods for improving the tensile strength of laminates without significantly impacting on their elastic modulus.

According to a first aspect, the present invention provides a method of preparing an elastomeric laminate article, the method comprising:
(i) providing a first aqueous coating composition comprising a first polymer and discrete silicon dioxide nanoparticles, wherein the first polymer is a polyurethane;
(ii) providing a second aqueous coating composition comprising a second polymer;
(iii) coating a substrate with the first aqueous coating composition to form a first film on the substrate;
(iv) coating the first film with the second aqueous coating composition to form a second film on the first film; and
(v) removing the substrate.

In an embodiment, the article is a condom. In an embodiment, the first aqueous coating composition is coated onto the substrate by dipping the substrate into the composition. When the substrate is a shaped former (such as a condom former), the method may comprise removing the laminate from the substrate to provide the formed article (such as a condom). In another embodiment, the laminated films may first be removed from the substrate and then formed into the article, for example by thermoforming.

Sequentially coating a substrate with multiple films allows for the thickness of the elastomer body to build up to a suitable level and for a consistent thickness to be obtained along the length of the condom or other article. However, the present inventors believe that the interface between the films constitutes a weakness in the structure. They have found that the incorporation of discrete silicon dioxide nanoparticles into at least the first film-forming composition, which includes a polyurethane, results in a condom having improved tensile strength compared with an equivalent condom lacking the discrete silicon dioxide nanoparticles, without a significant concomitant increase in elastic modulus. In particular, the present inventors have found that discrete silicon dioxide nanoparticles (unlike other forms of silica) are able to migrate to the interface of the first coated layer with ambient air, with only a limited quantity remaining dispersed within the bulk polymer matrix. This means that when the second film is formed on the first film, the silicon dioxide nanoparticles are present at the interface between the two films. Without wishing to be bound by theory, it is believed that the silicon dioxide nanoparticles present at the interface are able to provide reinforcement at the interface, which would otherwise constitute an inherent structural weakness, without significantly interfering with the properties of the bulk elastomer. As there is no significant accumulation of the silica in the bulk, the modulus, or the ability of the elastomer to withstand change when subject to strain is not compromised. As a result, the tensile strength of the condom is improved without a concomitant significant increase in elastic modulus. It is believed that the same principle applies to other laminate articles, especially thin ones.

According to a second aspect, the present invention provides an elastomeric laminate article (preferably a condom) obtained or obtainable by the method according to the first aspect.

According to a third aspect, the present invention provides an elastomeric laminate article which comprises a plurality of polymeric films and one or more interfaces, wherein each adjacent pair of films defines one of the interfaces,
wherein at least one of the plurality of polymeric films comprises a polyurethane,
wherein the elastomeric laminate article comprises silicon dioxide nanoparticles, and
wherein at least 60 wt % of the silicon dioxide nanoparticles are present at one or more of the interfaces defined at least in part by the polyurethane-comprising film.

According to a fourth aspect, the present invention provides a condom comprising a laminate, the laminate comprising a plurality of elastomeric films and one or more interfaces, wherein each adjacent pair of elastomeric films defines one of the interfaces,
wherein at least one of the plurality of elastomeric films comprises a polyurethane,
wherein the laminate comprises silicon dioxide nanoparticles, and
wherein at least 60 wt % of the silicon dioxide nanoparticles are present at one or
more of the interfaces defined at least in part by the polyurethane-comprising film.

According to a fifth aspect, the present invention provides a sealed package comprising a condom according to the second aspect or the fourth aspect.

The present invention will now be described further. In the following passages different aspects/embodiments of the invention are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The present invention provides a method of preparing an elastomeric laminate article. In an embodiment, the laminate article is a dipped good. The article may be for wearing in or on the body. In an embodiment, the article is a film to be used in contact with the human body (such as in contact with skin). The article may be for use in sexual activity, or for use during a medical procedure. Examples of laminate articles which may be made by the invention include condoms, dental dams, finger cots, gloves, and balloon catheters. In an embodiment, the laminate article is less than 1 mm thick, preferably less than 500 microns thick, most preferably less than 100 microns thick.

In an embodiment, the article is a condom. The condom is preferably a male condom, preferably one intended to cover substantially the entire penis. Alternatively, in some embodiments, the condom is a female condom.

The first step of the method comprises providing a first aqueous coating composition comprising a first polymer and discrete silicon dioxide nanoparticles. The first polymer is a polyurethane. The nature of the polyurethane is not particularly limited provided it can form a film having one or more of the mechanical properties (e.g. strength or flexibility) required for the article to be formed. Suitable polyurethanes for use as condom materials, for example, are known in the art. The polyurethane may be a polyether-based polyurethane, that is, a polyurethane derived from the polymerisation of a diisocyanate and a polyether. Alternatively, the polyurethane may be a polyester-based polyurethane, that is, a polyurethane derived from the polymerisation of a diisocyanate and a polyester. The polyurethane may be an aliphatic polyurethane, that is, a polyurethane comprising no aromatic structures. Alternatively, the polyurethane may be an aromatic polyurethane. The first polymer may be provided in a mixture with one or more further polyurethanes. It may be elastomeric, or a thermoplastic elastomer, but is preferably elastomeric.

Preferably, the first polymer is hydrophobic. Preferably, the first polymer has a logarithmic partition coefficient (logP) of greater than 1. The term “partition coefficient” is known in the art and refers to the octanol-water partition coefficient (P), that is, the ratio of distribution of the substance in a mixture of 1-octanol and water at equilibrium. The logarithmic partition coefficient is the logarithm to the base 10 of the partition coefficient. A logP of greater than 1 is indicative of hydrophobicity. Hydrophobic polymers may be advantageous in the present invention because they have less of a tendency than hydrophilic polymers to associate with the discrete silicon dioxide nanoparticles, and therefore do not interfere with the migration of the discrete silicon dioxide nanoparticles to interface with air during the coating step. As explained above, it is believed that the migration of the discrete silicon dioxide nanoparticles to the interface that ultimately enables the present invention to provide improved tensile strength without a concomitant significant increase in elastic modulus.

As mentioned above, the first aqueous coating composition comprises discrete silicon dioxide (“silica”) nanoparticles. By “nanoparticle” it is meant a particle having a diameter of from 1 to 1000 nm. By “discrete” it is meant that the particles are present in the first aqueous coating composition in an individualised or loosely associated form and are not fused together to form aggregates or agglomerates. In embodiments in which the particles are loosely associated in the aqueous coating composition, they are still considered discrete (rather than fused together to form aggregates or associates) because they can be separated again by stirring or dispersion. It is to be understood that the discrete silicon dioxide nanoparticles are amorphous, i.e. non-crystalline. Preferably, the discrete silicon dioxide nanoparticles are non-porous. Preferably, the discrete silicon dioxide nanoparticles are spherical or substantially spherical.

The discrete silicon dioxide particles used in the first aqueous coating composition are to be distinguished from other forms of silica. The preparation of fumed silica involves the hydrolysis of silicon tetrachloride vapour in a flame of hydrogen and oxygen. Molten particles of roughly spherical shapes are formed in the combustion process. These molten spheres of fumed silica, typically referred to as primary particles, fuse with one another by undergoing collisions at their contact points to form branched, three-dimensional chain-like aggregates. The force necessary to break aggregates is considerable and often considered irreversible because of the fusion. During cooling and collecting, the aggregates undergo further collision that may result in some mechanical entanglement to form agglomerates. Compared to the aggregates where the primary particles are fused together, agglomerates are thought to be loosely held together by Van der Waals forces and can be reversed, i.e. de-agglomerated, by proper dispersion in suitable media to form aggregates. However, these aggregates cannot be reversed to form primary particles because the primary particles are fused. As a result of the aggregation and agglomeration, fumed silica cannot be considered to be made up of discrete nanoparticles.

Another amorphous form of silica is precipitated silica, which is formed from a solution containing silicate salts. As with fumed silica, the primary particles of precipitated silica form aggregates (of fused primary particles) and agglomerates during the production process and therefore cannot be considered to be made up of discrete nanoparticles. Precipitated silica is a porous form of amorphous silica.

Preferably, the discrete silicon nanoparticles used in the first aqueous coating composition comprise surface silanol (Si—OH) groups. That is, silanol groups are preferably present on a surface of the nanoparticles. Due to the hydrophilic silanol groups on the surface and the hydrophobic Si—O—Si moieties in the silicon dioxide nanoparticle core, such silicon dioxide nanoparticles are amphiphilic. As a result, they are able to adsorb on the surface (air-water interface) of polymer-water droplets, helping them to migrate during the coating step to the interface of the first film with air rather than remaining within the bulk polymer matrix.

Preferably, the discrete silicon dioxide nanoparticles have a mean particle diameter of 100 nm or less, preferably 90 nm or less, 80 nm or less, 70 nm or less, 60 nm or less, 50 nm or less, 40 nm or less, 30 nm or less, 20 nm or less, or 10 nm or less. Preferably, they have a mean particle diameter from 1 to 100 nm, more preferably from 2 to 50 nm, still more preferably from 4 to 20 nm and most preferably from 5 to 10 nm. The mean particle diameter may, for instance, be measured by dynamic light scattering (DLS). Preferably, the mean particle diameter is a volume-weighted mean particle diameter (D_{4,3}). It is believed that small particle diameters are advantageous in the present invention, in part because of their increased ability to migrate to the interface of the coated layer with air during the coating step.

Preferably, the discrete silicon dioxide nanoparticles have a BET surface area of at least 200 m²/g, more preferably at least 250 m²/g, and most preferably at least 300 m²/g. It is believed that silicon dioxide nanoparticles having higher surface areas may provide greater improvements in tensile strength.

Preferably, the first aqueous coating composition comprises the first polymer in an amount of at least 10% by weight of the first aqueous coating composition, more preferably at least 15 wt %, at least 20 wt % or at least 25 wt %. Preferably, the first polymer is present in an amount of up to 60 wt %, up to 50 wt %, or up to 40 wt %, by weight of the first aqueous coating composition. In an embodiment, the first aqueous coating composition comprises the first polymer in an amount of from 10 to 50 wt % by weight of the first aqueous coating composition, more preferably from 20 to 40 wt %.

Preferably, the first aqueous coating composition comprises 0.1 to 3 wt % discrete silicon dioxide nanoparticles by weight of the first aqueous coating composition, more preferably from 0.2 to 4 wt %, still more preferably from 0.3 to 2 wt %, and more preferably from 0.5 to 1 wt %.

Preferably, the first aqueous coating composition comprises the first polymer and the discrete silicon dioxide nanoparticles in a weight ratio of the first polymer to the discrete silicon dioxide nanoparticles of from 5:1 to 200:1, more preferably from 10:1 to 200:1, still more preferably from 20:1 to 150:1, and most preferably from 40:1 to 80:1.

The first step of the method of the present invention preferably comprises combining a source of the first polymer with a source of discrete silicon dioxide nanoparticles to form the first aqueous coating composition. The source of the first polymer may be a dispersion of the first polymer in a liquid, preferably an aqueous dispersion. Thus, the source of the first polymer is preferably an aqueous polyurethane dispersion (“PUD”). Suitable PUDs for forming condoms and other articles are commercially available, such as Alberdingk® U228 from Alberdingk Boley.

Preferably, the source of discrete silicon dioxide nanoparticles may be a dispersion of discrete silicon dioxide nanoparticles in a liquid. Such dispersions are known in the art as “colloidal silica”. It is believed that by using colloidal silica rather than a solid form of silica aids with the dispersion of the nanoparticles and the migration of the nanoparticles to the interface. Preferably, the dispersion is an aqueous dispersion. Preferably, the pH of the aqueous dispersion is from 7 to 11, more preferably from 8 to 10. A suitable source of silicon dioxide nanoparticles for use in the present invention is Ludox® SM colloidal silica, which is an aqueous dispersion.

The first aqueous coating composition may further comprise other components that are typically included in polyurethane-based condom-forming materials. For instance, the first aqueous coating composition may further comprise a cross-linking agent, such as a carbodiimide cross-linking agent, preferably in an amount of from 0.2 to 5 wt %, more preferably from 0.5 to 2 wt % by weight of the first aqueous coating composition.

Alternatively or in addition, the first aqueous coating composition may further comprise a surfactant, preferably in an amount of from 0.05 to 1 wt % by weight of the first aqueous coating composition, more preferably from 0.1 to 0.5 wt %. It is believed that the silicon dioxide nanoparticles may cause an increase in surface tension of the first aqueous coating composition, making it more difficult to form an even layer on the substrate. The inclusion of a surfactant serves to reduce the surface tension of the first aqueous coating composition, making it easier to form an even layer on the substrate. Suitable surfactants to reducing the surface tension of aqueous coating compositions are known in the art and include, for instance, alkoxylated surfactants such as a polyether-modified siloxane surfactant.

The first aqueous coating composition preferably comprises from 40 to 90 wt % water by weight of the first aqueous coating composition, more preferably from 50 to 80 wt %, still more preferably from 60 to 75 wt %.

The second step of the method of the present invention comprises providing a second aqueous coating composition comprising a second polymer. The nature of the second polymer is not particularly limited provided it can form a film having one or more of the mechanical properties (e.g. strength or flexibility) required for the desired article, such as a condom. A number of suitable polymers are known in the art, such as polyurethanes, polyisoprenes, polyethylenes and copolymers of acrylonitrile and butadiene (“nitrile rubber”). Preferably, the second polymer is selected from a polyisoprene, a polyurethane or a mixture thereof. The polyisoprene may be synthetic cis-1,4-polyisoprene. Alternatively, the polyisoprene may be provided in the form of a natural rubber latex (“NRL”). Natural rubber latex typically comprises cis-1,4-polyisoprene together with small amounts of impurities, such as proteins, lipids, carbohydrates, inorganic salts and the like. Synthetic polyisoprene does not contain e.g. the allergenic proteins found in natural rubber latex and is therefore suitable for latex-sensitive users.

Most preferably, the second polymer is a polyurethane. In this embodiment, each of the optional or preferred features described above in relation to the first polymer are equally applicable to the second polymer.

The second polymer and the first polymer may be the same or different. For instance, the first polymer may be a polyurethane and the second polymer may be a polyisoprene or vice versa. As another example, the first polymer and the second polymer may be two different polyurethanes (e.g. having different molecular weights or monomer unit compositions). Preferably, however, the first polymer and the second polymer are the same (i.e the same polyurethane).

In some embodiments, the second aqueous coating composition may further comprise discrete silicon dioxide nanoparticles. As will be explained below, such embodiments are particularly preferred when the method further comprises forming a third film on the second film.

In embodiments in which the second aqueous coating composition further comprises discrete silicon dioxide nanoparticles, the second step of the method preferably comprises combining a source of the second polymer with a source of discrete silicon dioxide nanoparticles to form the second aqueous coating composition. The source of the second polymer may be a dispersion of the second polymer in a liquid, preferably an aqueous dispersion. When the second polymer is a polyurethane, the source of the second polymer is preferably an aqueous polyurethane dispersion (“PUD”), as described above. When the second polymer is a polyisoprene, the source of the second polymer is preferably an aqueous polyisoprene dispersion, such as an aqueous dispersion of synthetic cis-1,4-polyisoprene or a natural rubber latex. Suitable formulations of natural rubber latex and synthetic polyisoprene are known in the art. Each of the optional or preferred features described in relation to the “source of discrete silicon dioxide nanoparticles” described above in relation to the first aqueous coating composition applying equally to the second aqueous coating composition.

Each of the optional or preferred features described above in relation to the first coating composition and its constituent components are equally applicable to the second aqueous coating composition. Preferably, the first aqueous coating composition and the second aqueous coating composition are the same. For instance, the first aqueous coating composition and the second aqueous coating composition may constitute a single composition which is used in the coating steps described below.

The third step of the method comprises coating a substrate with the first aqueous coating composition to form a first film on the substrate. The substrate is later removed and does not form part of the finished article. In some embodiments, the substrate is a former. The term “former” is known in the art and in the case of a condom former refers to a condom-shaped mould to which a polymeric coating composition is applied to form a condom. Formers can, for instance, be made from glass, plastic or ceramic. In other embodiments, the substrate is a cast, plate or sheet. In an embodiment, the substrate is a glass, plastic or ceramic plate or sheet. In these embodiments, the first film is typically flat rather than taking the shape of the article to be formed.

The term “film” refers to a thin layer of polymeric material, the thickness of the layer typically being on the order of several microns or tens of microns, such as from 5 to 30 μm.

The third step preferably comprises applying a layer of the first aqueous coating composition to the substrate and drying the layer of the first aqueous coating composition to form the first film. Applying a layer of the first aqueous coating composition to the substrate may involve dipping the substrate into the first aqueous coating composition or spraying, brushing, casting or rolling the first aqueous coating composition onto the substrate. The layer of the first aqueous coating composition may be applied directly to the substrate, or, in some embodiments, a coagulant is applied to the substrate before the application of the first aqueous coating composition. In some embodiments, the substrate is stationary when the layer of the first aqueous coating composition is applied. In other embodiments, the substrate is in motion when the layer is applied. In embodiments in which the substrate is dipped into the first aqueous coating composition, varying the speed of dipping and/or withdrawal can be used to control the thickness of the layer. The drying step can be performed by evaporation in the open atmosphere or in an oven or evaporator. In some embodiments, the substrate is heated to facilitate drying.

As explained above, a significant proportion of the discrete silicon dioxide nanoparticles are able to migrate to the interface of the coating with air during the coating step. These silicon dioxide nanoparticles are fixed at the interface with air as the first film is formed. At this stage, at least some of the silicon dioxide nanoparticles present at the interface may loosely associate or cluster with each other. Other silicon dioxide nanoparticles present at the interface, or in the bulk of the polymer matrix, may remain in a fully individualised form. It will be appreciated that the silicon dioxide nanoparticles are still not fused together to form aggregates or agglomerates as described above in relation to fumed silica, even if some of them are loosely associated.

Preferably, the temperature and duration of the drying step is sufficient such that the first film has a water content of less than 10 wt %, more preferably less than 5 wt %. The temperature and duration of the drying step may vary depending on the constituents of the first aqueous coating composition (including the nature of the first polymer) and the thickness of the layer. The layer is preferably dried at a temperature of from 40 to 100° C., more preferably from 50 to 70° C., and/or for a period of from 1 to 10 minutes, more preferably from 3 to 7 minutes.

In some embodiments, the layer of the first aqueous coating composition undergoes a chemical change during the drying step. For instance, in embodiments in which the first aqueous coating composition comprises a cross-linking agent, cross-links between the polymer chains of the first polymer may form during the drying step.

In some embodiments, the first film has a thickness of from 5 to 30 μm. The preferred thickness of the first film will vary depending on the number of coating steps and the desired thickness of the final article which, in turn, will depend to some extent on the polymeric material(s) from which it is formed. For instance, polyurethane-based films may be suitably strong for use as condom materials at a lower thickness than polyisoprene-based films in view of their higher inherent strength. The first film, which comprises a polyurethane, preferably has a thickness of from 5 to 15 μm, more preferably from 6 to 10 μm.

The fourth step of the method of the invention comprises coating the first film with the second aqueous coating composition to form a second film on the first film. It is to be understood that the second aqueous coating composition is applied directly to the first film such that the second film and the first film are in direct contact, i.e. without any intervening films or layers. The fourth step preferably comprises applying a layer of the second aqueous coating composition to the first film and drying the layer of the second aqueous coating composition to form the second film. The first film preferably remains on the substrate for the duration of the fourth step. Thus, the fourth step preferably comprises dipping the film-coated substrate into the second aqueous coating composition or by spraying, brushing, casting or rolling the first aqueous coating composition onto the film-coated substrate. Aside from the temperature and duration of the drying step and the thickness of the resulting film, each of the optional or preferred features described above in relation to third step are equally applicable to the fourth step, except that the fourth step involves coating the first film rather than the substrate itself.

The temperature and duration of the step of drying the layer of the second aqueous coating composition to form the second film will depend on the nature of the second polymer. In embodiments in which the second polymer is a polyurethane, the layer is preferably dried at a temperature of from 40 to 100° C., more preferably from 50 to 70° C., and/or for a period of from 1 to 10 minutes, more preferably from 3 to 7 minutes. In embodiments in which the second polymer comprises a polyisoprene provided in the form of a natural rubber latex, the layer is preferably dried at a temperature of from 50 to 120° C., more preferably from 60 to 90° C., and/or for a period of from 30 seconds to 5 minutes, more preferably from 1 to 4 minutes. In embodiments in which the second polymer is a synthetic cis-1,4-polyisoprene, the layer is preferably dried at a temperature of from 70 to 130° C., more preferably from 80 to 120° C., and/or for a period of from 30 seconds to 5 minutes, more preferably from 1 to 4 minutes.

The preferred thickness of the second film will vary depending on the number of coating steps and the desired thickness of the final condom which, in turn, will depend to some extent on the polymeric material(s) from which it is formed. For instance, polyurethane-based films may be suitably strong for use as condom materials at a lower thickness than polyisoprene-based films in view of their higher inherent strength. When the second polymer is a polyurethane, the second film preferably has a thickness of from 5 to 15 μm, more preferably from 6 to 10 μm. When the second polymer is a polyisoprene, the second film preferably has a thickness of from 15 to 30 μm, more preferably from 20 to 25 μm.

In embodiments in which the first aqueous coating composition and the second aqueous coating composition are the same, the conditions of the third and fourth steps (e.g. temperature and duration) are preferably the same. Preferably, in embodiments in which the first aqueous coating composition and the second aqueous coating composition are the same, the thickness of the first film is substantially the same as, or is within 10% of, the thickness of the second film.

In some embodiments, the method further comprises providing a third aqueous coating composition comprising a third polymer, and, between the fourth step and the fifth step (discussed below) of the method of the invention, coating the second film with the third aqueous coating composition to form a third film on the second film. Each of the optional or preferred features described above in relation to second aqueous coating composition, coating step and film apply equally to the third aqueous coating composition, coating step and film. Preferably the first, second and third polymers (and preferably the first, second and third aqueous coating compositions) are the same. In other embodiments, at least two of the three polymers (or at least two of the three aqueous coating compositions) are the same and one is different. For instance, the first polymer may comprise a polyurethane, the second polymer may comprise a polyisoprene derived from a natural rubber latex, and the third polymer may comprise a polyurethane. Such an embodiment would result in the formation of a laminate PU-NRL-PU article, such as a condom. In other embodiments, all three of the polymers (or all three of the aqueous coating compositions) are different.

The number of coating steps is not particularly limited so long as the article does not become overly thick for its intended purpose. For instance, the method may further comprise providing a fourth aqueous coating composition comprising a fourth polymer, and, before the fifth step, coating the third film with the fourth aqueous coating composition to form a fourth film on the third film. The method may comprise one or more further successive steps of providing an aqueous coating composition and one or more further successive coating steps. In some embodiments, the aqueous coating composition used in each step (or at least the polymer used in each coating step) is the same. In some embodiments, the method comprises only three coating steps. In some embodiments, the method comprises only two coating steps.

In some embodiments, the method further comprises a step of heating the first film, the second film and, where present, the third film and any subsequent film on the substrate. Such a heating step, where present, occurs prior to the fifth step (discussed below) of the method of the invention. One purpose of such a heating step is to fully dry the films if they have not been fully dried by a previous drying step. The heating step may also induce or increase the formation of cross-links between the polymer chains present in the films. In one embodiment, the films are heated to a temperature of from 60 to 150° C., preferably to a temperature of from 80 to 140° C., and/or for a period of from 5 to 20 minutes, preferably from 8 to 15 minutes.

In an embodiment, a fifth step of the method of the invention comprises removing the substrate to provide the article; or removing the substrate to provide a laminate, and forming the article from the laminate. In embodiments in which the substrate is condom-shaped (e.g. a condom former), the fifth step provides a condom. In an embodiment in which the substrate is a cast, plate or sheet, the fifth step provides a laminate, which is then shaped to form a condom. In some such embodiments, the step of forming a condom from the laminate comprises heating the laminate and contacting the heated laminate with a mandril such that the laminate assumes the shape of the mandril. Examples of suitable means for shaping the laminate are disclosed in U.S. Pat. Nos. 4,576,156A and 5,458,936 A, the contents of which are incorporated by reference. In some embodiments, the fifth step involves stripping the film structure from the substrate. In some embodiments, the film structure is leached (e.g. in alkaline solution at a temperature of from 20 to 40° C.) before the substrate is removed to provide the laminate.

In embodiments of the invention in which the article is a condom, the condom preferably has a thickness of from 10 to 60 um. By “thickness” it is meant the wall thickness (i.e. the combined thickness of the films as defined herein that form the condom), not including any bead formed at the opening of the condom. The preferred thickness of the condom will vary in part based on the polymeric material(s) from which it is formed. Preferably, the condom has a thickness of from 10 to 30 μm, or from 10 to 25 μm, or from 10 to 20 μm. The increase in tensile strength provided by the discrete silicon nanoparticles of the present invention allows for relatively condom thicknesses to be used.

Preferably, the article (preferably the condom) has a tensile strength of at least 10 MPa, or at least 20 MPa, or at least 30 MPa, or at least 40 MPa. In some embodiments, the article (preferably the condom) has a tensile strength of at most 100 MPa, or at most 90 MPa, or at most 80 MPa. The term “tensile strength” refers to the maximum stress taken by the article before breaking.

Preferably, the article (preferably the condom) has an elastic modulus at 300% elongation of at most 5 MPa, or at most 4 MPa, or at most 3.5 MPa. In these embodiments, the article (preferably the condom) may have an elastic modulus at 300% elongation of at least 2 MPa, or at least 2.5 MPa, or at least 3 MPa. The term “elastic modulus” refers to the stress at a particular elongation of the article and relates to the hardness of the material. Lower elastic modulus articles are softer and are typically perceived to have better feel.

Preferably, the article (preferably the condom) has an elastic modulus at 500% elongation of at most 8 MPa, or at most 7 MPa, or at most 6 MPa, or at most 5.5 MPa. In these embodiments, the article (preferably the condom) may have an elastic modulus at 500% elongation of at least 2.5 MPa, or at least 3 MPa.

Preferably, the tensile strength and elastic moduli are determined in accordance with the procedures described in Example 2. Preferably, the tensile strength and elastic moduli are determined in accordance with ISO standard ISO 4074:2015

Preferably, the article (preferably the condom) has a tensile strength at least 10% higher, more preferably at least 20% higher, still more preferably at least 30% higher, than that of a article (preferably a condom) prepared by the same method but without using discrete silicon dioxide nanoparticles.

Preferably, the article (preferably the condom) has an elastic modulus at 300% elongation of within 10%, preferably within 5%, of that of an article (preferably a condom) prepared by the same method but without using discrete silicon dioxide nanoparticles.

Preferably, the article (preferably the condom) has an elastic modulus at 500% elongation of within 10%, preferably within 5%, of that of an article (preferably a condom) prepared by the same method but without using discrete silicon dioxide nanoparticles.

In some embodiments in which the article is intended to be worn on the body, such as a condom, glove or finger cot (preferably a condom), the method further comprises coating one or more surfaces of the article with a finishing powder. The one or more surfaces may be an inner and/or an outer surface, preferably an inner surface and an outer surface. “Inner” in the context of a male condom refers to the penis-facing side, whereas “outer” refers to the side facing the user's partner; “inner” in the context of a glove or finger cot refers to the finger-facing side. In embodiments in which the method further comprises coating one or more surfaces of the condom with a finishing powder, this is done prior to rolling, and before any lubricant is applied. The step of coating one or more surfaces of the article with a finishing powder may comprise applying the finishing powder to the one or more surfaces as a powder or as a liquid slurry (preferably an aqueous slurry). In the latter embodiment, the water is allowed to evaporate to form the coating of the finishing powder on the one or more surfaces of the article. Finishing powders are known in the art. They are typically alkaline, and are typically based on compounds such silica, talc, carbonates, corn starch and the like. They are used to prevent the surfaces of the article from sticking to each other, and to assist with donning. In particular, including a finishing powder on an inner surface of a condom serves to prevent the condom from sticking to itself, while including a finishing powder on an outer surface also serves to prevent the condom from sticking to other condoms during production.

In some embodiments, the method further comprises applying a dose of a lubricant to one or more surfaces of the condom to form a lubricated condom. The one or more surfaces may be an inner surface and/or an outer surface of the condom. Preferably, a dose of a lubricant is applied to at least the outer surface of the condom. Suitable lubricants for condoms are known in the art and are typically water-based or silicone oil-based. In some embodiments, the condom is rolled prior to the application of the lubricant. In this embodiment, the dose of lubricant may be applied at or near a tip of the rolled condom. The lubricant may then migrate along the rolls of the condom over time (including after the condom is sealed within a package, as discussed below). In this embodiment, the condom may already be within the package, or on a material which will form a part of the package (such as one piece of foil), at the time the dose of lubricant is applied. In embodiments in which the condom is sealed within a package (as described below), only the sealing step must necessarily take place after the lubricant is applied.

Alternatively, a dose of a lubricant may be applied to the condom prior to any rolling step. The lubricant may be applied in a variety of known ways, for example, by spraying, rolling over a sponge soaked with the lubricant, or the dose of lubricant may be applied to one or more spots along a length of the condom prior to rolling. It will be appreciated that if the lubricant is pre-applied to the condom in all or substantially all of the area where it is required, such that it does not need to migrate to the desired area, a higher viscosity of the lubricant may be tolerated.

The method may comprise further steps, such as electrically testing the condom for holes, rolling the condom and/or sealing the condom within a package. Suitable sealed packages for condoms are known in the art and may include, for instance, two sheets of a laminate material sealed along their edges around the condom. The laminate material may, for instance, include a layer of aluminium. Another possible sealed package is a plastic pot sealed with a film lid, or so-called “butter dish”. The condom is preferably provided within the package in a rolled state.

According to a second aspect, the present invention provides an article obtained or obtainable by the method according to the first aspect, preferably a condom.

According to a third aspect, the present invention provides an elastomeric laminate article which comprises a plurality of polymeric films and one or more interfaces, wherein each adjacent pair of films defines one of the interfaces,
wherein at least one of the plurality of polymeric films comprises a polyurethane,
wherein the elastomeric laminate article comprises silicon dioxide nanoparticles, and
wherein at least 60 wt % of the silicon dioxide nanoparticles are present at one or more of the interfaces defined at least in part by the polyurethane-comprising film.

Optionally, the article is a condom.

By “laminate” it is meant a composite structure made up of a plurality of layers, which in the present invention preferably constitute a plurality of elastomeric films. The number of films is not particularly limited and will depend, in part, on the desired thickness of the article. Preferably, however, the laminate comprises only two or three films. In some embodiments, the laminate consists of the plurality of films. It is to be understood that the laminate forms the wall(s) of the article. In the case of a male condom, the condom has an inner surface (i.e. the penis-facing side in use), and an outer surface (i.e. the side facing the user's partner in use), the surfaces of the laminate defining the inner and outer surfaces.

It is to be understood that each of the films comprises a polymer, preferably an elastomer (a rubber-like polymer with elastic properties). The nature of the elastomer is not particularly limited provided it can form an elastomeric film having one or more of the mechanical properties (e.g. strength or flexibility) required for a condom or other article to be made. Suitable polymers for use in the other films (preferably elastomeric films) include polyurethanes, polyisoprenes, polyethylenes and copolymers of acrylonitrile and butadiene (“nitrile rubber”), as described in relation to the first aspect. It is preferred that an elastomeric film defining an inner surface of a condom comprises a polyurethane.

The polymer may be the same in each of the films. For instance, each of the films may comprise the same polyurethane. In some embodiments, the polymer is the same in some of the films but not others. For instance, the laminate may comprise alternating films comprising a polyurethane and a polyisoprene respectively. Alternatively, the laminate may comprise alternating films comprising different polyurethanes (e.g. having different molecular weights or monomer unit compositions). In other embodiments, the polymer is different in each of the films. Preferably, each of the plurality of layers comprises a polyurethane, preferably the same polyurethane. The polymer present in one or more of the films of the third aspect (preferably each of the films of the third aspect) is preferably cross-linked.

As mentioned above, the laminate comprises silicon dioxide nanoparticles. The silicon dioxide nanoparticles are not necessarily “discrete” as defined above in relation to the aqueous coating compositions of the first aspect. This is because at least some of the silicon dioxide nanoparticles present at the one or more interfaces within the laminate may loosely associate or cluster with each other, and cannot, in the laminate, be separated again to form fully individualised particles. Other silicon dioxide nanoparticles present at the one or more interfaces, or in the bulk of the films, may remain in an individualised form. It will be appreciated that the silicon dioxide nanoparticles in the laminate of the third aspect are still not fused together to form aggregates or agglomerates (as described above in relation to fumed silica), even if some of them are loosely associated.

At least 60 wt % of the silicon dioxide nanoparticles are present at one or more of the interfaces defined at least in part by a polyurethane-containing film, more preferably at least 70 wt %, and most preferably at least 80 wt %. In other words, at most 40 wt %, more preferably at most 30 wt %, and most preferably at most 20 wt % of the silicon dioxide nanoparticles are not present at one or more of the interfaces (i.e. they are present within the “bulk” of the films). As mentioned above, each interface is defined by an adjacent pair of films. It will be appreciated that the films forming a pair must be in direct contact with one another in order to define an interface. The term “interface”, as used herein, refers to a region extending from the boundary between two adjacent films to a distance of 500 nm, preferably 100 nm, or 50 nm, or 20 nm, or 10 nm either side of and orthogonal to the boundary. The term “bulk” refers to the portions of the films that do not form an interface. The proportion of silicon dioxide nanoparticles present at the one or more interfaces defined at least in part by a polyurethane-containing film can be determined by scanning electronic microscopy. In some embodiments, at most 95 wt %, or at most 90 wt %, of the silicon dioxide nanoparticles are present at one or more of the interfaces defined at least in part by a polyurethane-containing film. In other words, in some embodiments at least 5 wt %, or at least 10 wt % of the silicon dioxide nanoparticles are not present at one or more of the interfaces defined at least in part by a polyurethane-containing film. Silicon dioxide nanoparticles that are present within the bulk of the films may be dispersed within the polymer matrix, and/or may be present at one or more defects within the films.

As explained above, the present inventors have found that by concentrating silicon dioxide nanoparticles at the interface between an adjacent pair of films in preference to the bulk, it is possible to reinforce the structural weakness associated with the interface without significantly interfering with the properties of the bulk polymer. It is possible to do this when at least one of the films comprises a polyurethane. As a result, the tensile strength of the article may be improved without a concomitant significant increase in elastic modulus.

Preferably, the silicon nanoparticles comprise surface silanol (Si—OH) groups. That is, silanol groups are preferably present on the surface of the nanoparticles. Due to the hydrophilic silanol groups on the surface and the hydrophobic Si—O—Si moieties in the silicon dioxide nanoparticle core, such silicon dioxide nanoparticles are amphiphilic. This is one means of increasing the concentration of the silicon dioxide nanoparticles at the one or interfaces relative to the bulk. This is because, during the preparation of the laminate, the amphiphilic silicon dioxide nanoparticles are able to adsorb on the surface (air-water interface) of polymer-water droplets, helping them to migrate to the one or more interfaces rather than remaining within the bulk polymer matrix.

Preferably, the silicon dioxide nanoparticles have a primary particle diameter of from 1 to 100 nm, more preferably from 2 to 50 nm, still more preferably from 4 to 20 nm and most preferably from 5 to 10 nm. By “primary particle diameter” it is meant the diameter of the individual silicon dioxide nanoparticles, rather than the size of any associations or clusters of the nanoparticles.

Preferably, the silicon dioxide nanoparticles have a BET surface area of at least 200 m²/g, more preferably at least 250 m²/g, and most preferably at least 300 m²/g.

Preferably, the laminate comprises at least 2 wt %, at least 3 wt %, at least 4 wt %, at least 5 wt %, or at least 6 wt % silicon dioxide nanoparticles by weight of the laminate. Preferably, the laminate comprises at most 10 wt % silicon dioxide nanoparticles by weight of the laminate.

Preferably, one or more of the films (preferably each film) has a thickness of from 5 to 30 μm. The optional or preferred thicknesses described above in relation to the films of the first aspect apply equally to the films of the third aspect.

Preferably, the thickness of the article and its mechanical properties (tensile strength, elastic modulus etc.) are as defined in relation to the first aspect. Preferably, the article has a tensile strength at least 10% higher, more preferably at least 20% higher, still more preferably at least 30% higher, than that of an article that is otherwise identical but does not comprise silicon dioxide nanoparticles.

Preferably, the article has an elastic modulus at 300% elongation of within 10%, preferably within 5%, of that of an article that is otherwise identical but does not comprise silicon dioxide nanoparticles.

Preferably, the article has an elastic modulus at 500% elongation of within 10%, preferably within 5%, of that of an article that is otherwise identical but does not comprise silicon dioxide nanoparticles.

According to a fourth aspect, the present invention provides a condom comprising a laminate, the laminate comprising a plurality of elastomeric films and one or more interfaces, wherein each adjacent pair of elastomeric films defines one of the interfaces,
wherein at least one of the plurality of elastomeric films comprises a polyurethane,
wherein the laminate comprises silicon dioxide nanoparticles, and
wherein at least 60 wt % of the silicon dioxide nanoparticles are present at one or more of the interfaces that are defined at least in part by the polyurethane-comprising film.

Each of the optional or preferred features described in relation to the polyurethane of the first aspect applies equally to the polyurethane of the third and fourth aspects, except where the context otherwise requires. Each of the optional or preferred features described in relation to the successive polymers of the first aspect (“first polymer”, “second polymer” etc.) apply equally to the successive polymers of the films of the third and fourths aspects, except where the context otherwise requires. In embodiments in which the polymer present in one or more of the films is a polyisoprene derived from a natural rubber latex, the film(s) in which it is present are considered to be “natural rubber latex films”.

In some embodiments (of the fourth aspect, or where the article is a condom in the case of the third aspect) the condom further comprises, on one or more surfaces thereof, a finishing powder. The one or more surfaces may be an inner and/or an outer surface, preferably an inner surface and an outer surface. Suitable finishing powders and means for applying them are disclosed in relation to the first aspect.

In some embodiments (of the fourth aspect, or where the article is a condom in the case of the third aspect) the condom further comprises, on one or more surfaces thereof, a lubricant. The one or more surfaces may be an inner surface and/or an outer surface of the condom, preferably at least the outer surface. Suitable lubricants are described in relation to the first aspect.

According to a fifth aspect, the present invention provides a sealed package comprising a condom according to the second, third or fourth aspect. The sealed package is preferably as described in relation to the first aspect.

A number of especially preferred embodiments of the first and fourth aspects of the invention will now be described.

In certain preferred embodiments, the present invention provides a method of preparing a condom, the method comprising:
(i) providing an aqueous coating composition comprising a polyurethane and discrete silicon dioxide nanoparticles;
(ii) coating a substrate with the aqueous coating composition to form a first film on the substrate;
(iii) coating the first film with the aqueous coating composition to form a second film on the first film;
(iv) optionally coating the second film with the aqueous coating composition to form a third film on the second film; and
(v) removing the substrate to provide a condom; or removing the substrate to provide a laminate, and forming a condom from the laminate.

In certain preferred embodiments, the present invention provides a condom comprising a laminate, the laminate comprising N elastomeric films and N−1 interfaces, wherein each adjacent pair of elastomeric films defines one of the interfaces, wherein N is at least 2, preferably from 2 to 4,
wherein each elastomeric film comprises a polyurethane, preferably the same polyurethane,
wherein the laminate comprises silicon dioxide nanoparticles, and
wherein at least 60 wt % of the silicon dioxide nanoparticles are present at one or more of the interfaces.

In certain preferred embodiments, the present invention provides a condom comprising a laminate, the laminate comprising two elastomeric films defining an interface therebetween,
wherein each elastomeric film comprises a polyurethane, preferably the same polyurethane,
wherein the laminate comprises silicon dioxide nanoparticles, and
wherein at least 60 wt % of the silicon dioxide nanoparticles are present at the interface.

In certain preferred embodiments, the present invention provides a condom comprising a laminate, the laminate comprising a first elastomeric film, a second elastomeric film and a third elastomeric film,
wherein the first elastomeric film and the second elastomeric film define a first interface, and the second elastomeric film and the third elastomeric polyurethane film define a second interface,
wherein each elastomeric film comprises a polyurethane, preferably the same polyurethane,
wherein the laminate comprises silicon dioxide nanoparticles,
wherein at least 60 wt % of the silicon dioxide nanoparticles are present at the first interface or the second interface.

In each of the foregoing especially preferred embodiments, each film preferably has the same chemical composition.

The present invention will now be described in relation to the following non-limiting figures.

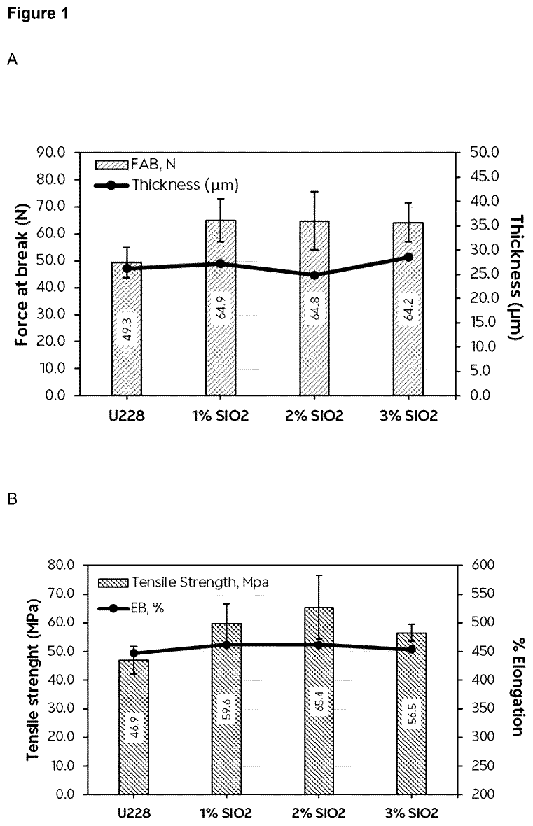

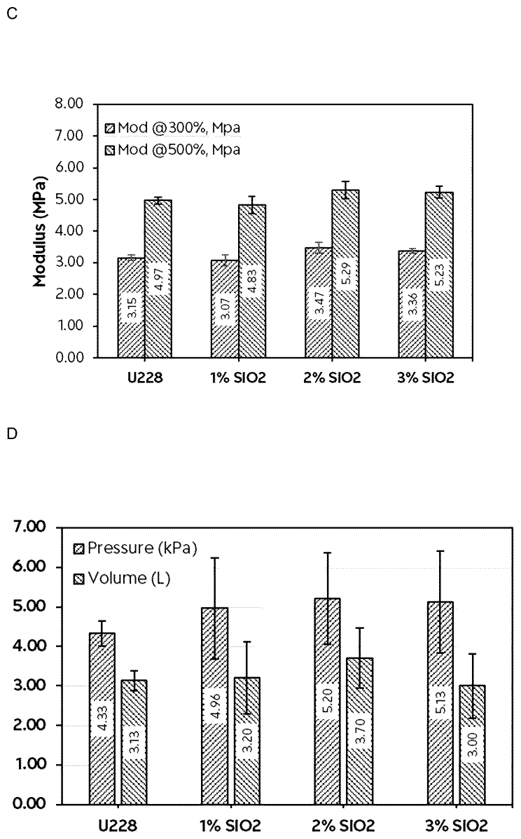

FIG. 1 shows the tensile and burst properties of the condoms prepared in accordance with Example 1, as measured in accordance with Example 2. In each graph, the data points from left to right are for the control formulation (U228), 1 wt % SiO₂, 2 wt % SiO₂ and 3 wt % SiO₂.

In FIG. 1A, the bars represent force at break (N, left hand axis) and the lines represent thickness (μm, right hand axis).

In FIG. 1B, the bars represent tensile strength (MPa, left hand axis) and the lines represent elongation at break (%, right hand axis).

In FIG. 1C, the left hand bars represent modulus at 300% elongation (MPa) and the right hand bars represent modulus at 500% elongation (MPa).

In FIG. 1D, the right hand bars represent burst volume (L) and the left hand bars represent burst pressure (kPa).

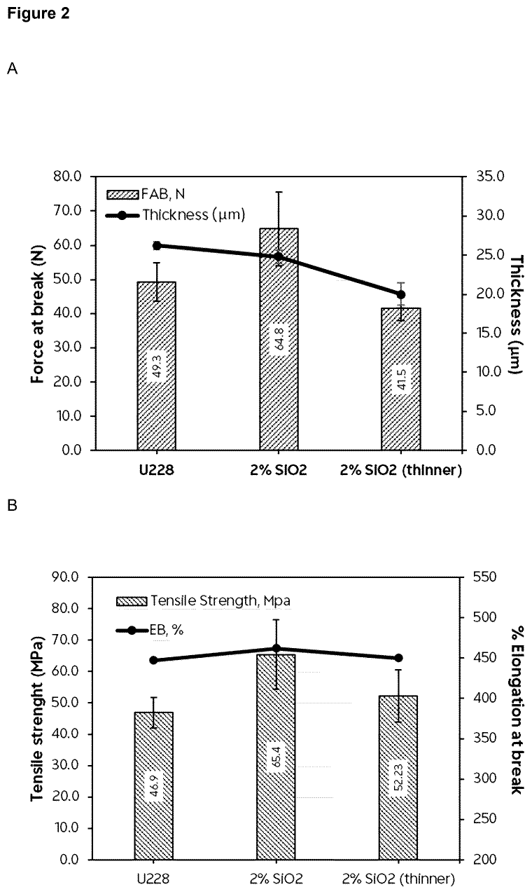

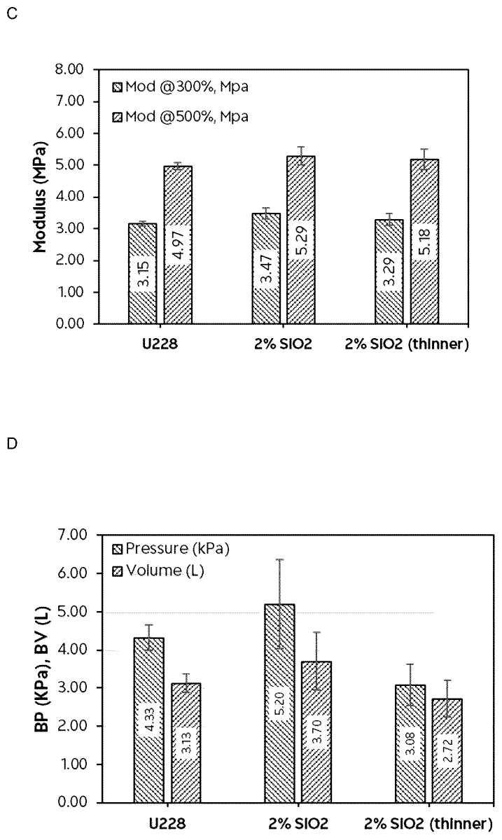

FIG. 2 shows the tensile and burst properties of the condom prepared in accordance with Example 3, as measured in accordance with the methods disclosed in Example 2 and compared with the 2 wt % SiO₂ and control condoms of Example 1. In each graph, the data points from left to right are for the control formulation (U228) at 26.3 μm wall thickness, 2 wt % SiO₂ at 24.8 μm wall thickness and 2 wt % SiO₂ at 20.0 μm wall thickness.

In FIG. 2A, the bars represent force at break (N, left hand axis) and the line represents thickness (μm, right hand axis).

In FIG. 2B, the bars represent tensile strength (MPa, left hand axis) and the line represents elongation at break (%, right hand axis).

In FIG. 2C, the left hand bars represent modulus at 300% elongation (MPa) and the right hand bars represent modulus at 500% elongation (MPa).

In FIG. 2D, the right hand bars represent burst volume (L) and the left hand bars represent burst pressure (kPa).

The present invention will now be described in relation to the following non-limiting Examples.

### EXAMPLE 1

An aliphatic polyether polyurethane aqueous dispersion (Alberdingk® U228 (50% solids), commercially available from Alberdingk Boley) was diluted with deionised water to 35% solids and mixed with a water-based carbodiimide cross-linking agent (Carbodilite® SV-02 (40% solids), commercially available from Nisshinbo Chemical Inc.) in an amount of 3 wt % solid carbodiimide based on the total solids content of the diluted polyurethane dispersion. The resulting mixture was stirred for 30 minutes. Then, Ludox® SM colloidal silica with a surface area of 363 m²/g (a 30 wt % suspension of SiO₂ nanoparticles in H₂O, commercially available from Sigma-Aldrich) was added in an amount of 1 wt %, 2 wt % or 3 wt % solids based on the weight of solid polyurethane, and the resulting mixture was stirred for 30 minutes and then sonicated for 30 minutes. A liquid polyether-modified siloxane surfactant (BYK-348, commercially available from BYK) was added in an amount of 0.2% w/v and the resulting mixture was stirred for 30 minutes.

A condom was prepared by dipping a glass former into the mixture three times to form three layers of material on the former using a dipping robot. Between each dipping step, the material was dried on the former at 60° C. for 5 minutes in a circulating air oven to form a film. After the final film-forming step, the material was dried on the former at 120° C. for 12 minutes in another circulating air oven. After cooling down to room temperature, a starch-based finishing slurry was applied and the condom was stripped from the glass former before drying in a dryer. The three-layered condom had a wall thickness of 25-30 μm.

A control was prepared in the same manner as above but without any added colloidal silica or surfactant.

The sample formulations are summarised in the following table:

TABLE 1
Formulations used to prepare condoms of Example 1

| | U228 | SV-02 | SM-SiO₂ | BYK |
|---|---|---|---|---|
| Condom | (phr) | (wt %) | (wt %) | (% w/v) |
| Control (U228) | 100 | 3.0 | 0 | 0 |
| 1 wt % SiO₂ | 100 | 3.0 | 1.0 | 0.2 |
| 2 wt % SiO₂ | 100 | 3.0 | 2.0 | 0.2 |
| 3 wt % SiO₂ | 100 | 3.0 | 3.0 | 0.2 |

The addition of colloidal silica was found to have no noticeable effect on the appearance of the condoms. All condoms appeared transparent after removal of the starch-based finishing powder.

### EXAMPLE 2

The tensile (force at break, tensile strength, elongation at break, modulus) and air burst (burst pressure and burst volume) properties of condom samples obtained from condoms prepared in accordance with Example 1 were determined.

To determine the tensile properties, a ring-shaped sample was cut from each condom before being tested with a universal tensile tester in accordance with ISO 4074. The samples were tested with a 500 N load cell and stretched at a rate of 500 mm/min until break. The tensile stress was calculated by finding the ratio of force and the initial cross-sectional area of the specimen. The strain or elongation was defined as the ratio of the elongated length to the initial length of the sample. The moduli was defined as the stress at specific elongation e.g. modulus at 300% (M300) and 500% (M500) elongation are referred to as the stress at 300% and 500% elongation, respectively. The force, elongation and stress at breaking were defined as the force at break, elongation at break and tensile strength, respectively. Five replicate measurements for each sample were used to calculate mean average values for all properties.

The thickness of each condom (i.e. the wall thickness, excluding the bead) was measured at a right angle to the length of the condom, when it is unrolled and laid flat without any creases. Three thickness measurements were made for each sample condom by a thickness gauge, and a mean average was determined.

Burst pressure and burst volume of condom samples were assessed by inflating the condom like a balloon to measure the air pressure and air volume respectively needed to burst it according to ISO 23409:2011. The condom was unrolled and clamped to a stem, leaving about 150 mm to be inflated. The testing apparatus inflated the condom with a clean, oil-free and moisture-free air at a specified rate.

The results are shown in Table 2 and FIGS. 1A to 1D.

TABLE 2
Tensile and burst properties of PU condom produced in accordance
with Example 1 with SM-SiO₂ 0, 1, 2 and 3 wt %

| Properties | Control PU | 1% SiO₂-SM | 2% SiO₂-SM | 3% SiO₂-SM |
|---|---|---|---|---|
| Thickness (μm) | 26 ± 1 | 27 ± 1 | 25 ± 1 | 29 ± 4 |
| M300 (MPa) | 3.15 ± 0.09 | 3.07 ± 0.16 | 3.47 ± 0.17 | 3.36 ± 0.08 |
| M500 (MPa) | 5.00 ± 0.11 | 4.83 ± 0.27 | 5.30 ± 0.27 | 5.23 ± 0.18 |
| Force at break (N) | 49.25 ± 5.61 | 64.9 ± 7.9 | 64.8 ± 10.7 | 64.2 ± 7.3 |
| Tensile strength | 46.93 ± 4.88 | 59.62 ± 6.86 | 65.42 ± 11.02 | 56.47 ± 2.4 |
| (MPa) | | | | |
| Elongation at break | 447 ± 5 | 462 ± 8 | 462 ± 8 | 453 ± 8 |
| (%) | | | | |
| Burst pressure (KPa) | 4.33 ± 0.32 | 4.96 ± 1.282 | 5.2 ± 1.16 | 5.125 ± 1.29 |
| Burst volume (L) | 3.13 ± 0.25 | 3.20 ± 0.91 | 3.70 ± 0.75 | 3.00 ± 0.816 |

The addition of SiO₂ nanoparticles to the formulation was found to significantly increase the force at break and tensile strength of the condom samples. However, there was only a limited difference in modulus at 300% elongation, modulus at 500% elongation, elongation at break, burst pressure or burst volume as a result of the addition of SiO₂ nanoparticles. It should be noted that the tensile strength peaked at 2 wt % SiO₂. It is believed that increasing the SiO₂ further increased the ductility of the material, ultimately reducing its tensile strength. It will be appreciated, however, that the tensile strength of the 3 wt % SiO₂ condom was still significantly higher than the control.

### EXAMPLE 3

A further condom was prepared using the 2 wt % SiO₂ formulation of Example 1 by the same method disclosed in Example 1 but having a lower wall thickness than the 2 wt % SiO₂ condom of Example 1 (20 μm as opposed to 25 μm). The physical properties were measured in accordance with Example 2.

The results are shown in Table 3 and FIGS. 2A to 2D. The thicker 2% SiO₂ condom and control condom from Examples 1 and 2 have been included for comparison.

TABLE 3
Tensile and burst properties of PU condom produced in accordance
with Example 3 compared with reference condoms from Example 1

| | Control PU | 2% SiO₂-SM | 2% SiO₂-SM |
|---|---|---|---|
| Properties | (Example 1) | (Example 1) | (Example 3) |
| Thickness (μm) | 26 ± 1 | 25 ± 1 | 20 ± 1 |
| M300 (MPa) | 3.15 ± 0.09 | 3.47 ± 0.17 | 3.29 ± 0.19 |
| M500 (MPa) | 5.00 ± 0.11 | 5.30 ± 0.27 | 5.18 ± 0.33 |
| Force at break (N) | 49.25 ± 5.61 | 64.8 ± 10.7 | 41.5 ± 3.49 |
| Tensile strength (MPa) | 46.93 ± 4.88 | 65.42 ± 11.02 | 52.23 ± 8.25 |
| Elongation at break (%) | 447 ± 5 | 462 ± 8 | 450 ± 14 |
| Burst pressure (KPa) | 4.33 ± 0.32 | 5.2 ± 1.16 | 3.08 ± 0.55 |
| Burst volume (L) | 3.13 ± 0.25 | 3.70 ± 0.75 | 2.72 ± 0.48 |

The 20 μm thickness condom sample at 2 wt % SiO₂ had a lower force at break than both the equivalent sample at 25 μm thickness and the control sample at 26 μm thickness. However, it was still within the acceptable range for a condom. Moreover, the tensile strength of the 20 um thickness condom sample at 2 wt % SiO₂ was higher than the control sample, while the modulus at 300% elongation and the modulus at 500% elongation were comparable. The burst pressure for the 20 μm thickness condom sample at 2 wt % SiO₂ was slightly lower than for the other two samples, but still within the acceptable range.

Overall, the data demonstrates that the inclusion of SiO₂ nanoparticles in the formulation allows for the thickness of the condom to be reduced while maintaining acceptable physical properties.

### EXAMPLE 4

Scanning electron microscopy (SEM) images were taken of samples cut from condoms prepared in accordance with Example 1 to investigate the surface morphology of condom samples. Each sample was cut into small pieces and then immersed in liquid nitrogen for 1 minute before immediate breaking the sample to prepare a cross-sectioned sample. The sample was placed onto a sample stub before coating by gold under vacuum. Finally, the sample grid was subjected to surface micrograph analysis using a field emission scanning election microscope (FE-SEM).

SEM micrographs with magnitude of 5000×, 10000× and 25000× were taken. For the SiO₂-containing samples, SiO₂ nanoparticles were visible at the interfaces between the polyurethane layers. This provides evidence of the ability of the SiO₂ nanoparticles to migrate to the air-water interface during each coating step and to remain at the interface as each successive film is formed. At 1 wt % SiO₂, the concentration of SiO₂ nanoparticles dispersed in the bulk polyurethane matrix is very low. At 2 wt % and 3 wt % SiO₂, the concentration of SiO₂ nanoparticles dispersed in the bulk polyurethane matrix increases.

### EXAMPLE 5

Scanning electron microscopy (SEM) with energy dispersive x-ray analysis (EDX) was performed on the samples imaged in Example 4 to investigate the elements distributed in the polyurethane matrix.

The condom sample without SiO₂ mainly contained C and O as the main components of polyurethane. However, a Si component could be observed in the samples with SiO₂, indicating the presence of SiO₂. The SEM-EDX micrographs clearly showed that Si-containing particles are predominantly at the interface between the polyurethane layers. The SEM-EDX micrographs provide further evidence that discrete SiO₂ nanoparticles are able to migrate to the interfaces between the films resulting from each dipping step.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents.

1. A method of preparing an elastomeric laminate article, the method comprising:
(i) providing a first aqueous coating composition comprising a first polymer and discrete silicon dioxide nanoparticles, wherein the first polymer is a polyurethane;
(ii) providing a second aqueous coating composition comprising a second polymer;
(iii) coating a substrate with the first aqueous coating composition to form a first film on the substrate;
(iv) coating the first film with the second aqueous coating composition to form a second film on the first film; and
(v) removing the substrate.

2. The method according to claim 1, wherein the second polymer is selected from a polyisoprene, a polyurethane and a mixture thereof.

3. The method according to claim 1, wherein the second polymer is a polyurethane.

4. The method according to claim 1, wherein the discrete silicon dioxide nanoparticles have a particle diameter of from 1 to 100 nm.

5. The method according to claim 1, wherein the discrete silicon dioxide nanoparticles have a BET surface area of at least 200 m²/g.

6. The method according to claim 1, wherein the discrete silicon dioxide nanoparticles comprise surface silanol groups.

7. The method according to claim 1, wherein the step of providing a first aqueous coating composition comprising a first polymer and discrete silicon dioxide nanoparticles comprises combining a source of the first polymer with a source of discrete silicon dioxide nanoparticles to form the first aqueous coating composition.

8. The method according to claim 7, wherein the source of the first polymer comprises an aqueous polyurethane dispersion.

9. The method according to claim 1, wherein the method further comprises providing a third aqueous coating composition comprising a third polymer, and, between the step of coating the first film with the second aqueous coating composition to form a second film on the first film and the step of removing the substrate, coating the second film with the third aqueous coating composition to form a third film on the second film.

10. The method according to claim 1, wherein the elastomeric laminate article is a condom.

11. (canceled)

12. An elastomeric laminate article which comprises a plurality of polymeric films and one or more interfaces, wherein each adjacent pair of films defines one of the interfaces,
wherein at least one of the plurality of polymeric films comprises a polyurethane,
wherein the laminate comprises silicon dioxide nanoparticles, and
wherein at least 60 wt % of the silicon dioxide nanoparticles are present at one or more of the interfaces defined at least in part by a polyurethane-comprising film.

13. A condom comprising a laminate, the laminate comprising a plurality of elastomeric films and one or more interfaces, wherein each adjacent pair of elastomeric films defines one of the interfaces,
wherein at least one of the plurality of elastomeric films comprises a polyurethane,
wherein the laminate comprises silicon dioxide nanoparticles, and
wherein at least 60 wt % of the silicon dioxide nanoparticles are present at one or more of the interfaces that are defined at least in part by the polyurethane-comprising film.

14. The elastomeric laminate article according to claim 12, wherein at least 70 wt % of the silicon dioxide nanoparticles are present at one or more of the interfaces, more preferably at least 80 wt %.

15. (canceled)

16. The method according to claim 3, wherein the first polymer and the second polymer are the same polyurethane.

17. The method according to claim 4, wherein the discrete silicon dioxide nanoparticles have a particle diameter of from 2 to 50 nm.

18. The method according to claim 5, wherein the discrete silicon dioxide nanoparticles have a BET surface area of at least 250 m²/g.

19. The method according to claim 7, wherein the source of discrete silicon dioxide nanoparticles is colloidal silica.

20. The condom according to claim 13, wherein at least 70 wt % of the silicon dioxide nanoparticles are present at one or more of the interfaces.

21. The method according to claim 1, wherein the first aqueous coating composition comprises the discrete silicon dioxide nanoparticles in an amount of 0.1 to 3 wt %.

22. The method according to claim 1, wherein the first aqueous coating composition comprises the first polymer and the discrete silicon dioxide nanoparticles in a weight ratio of the first polymer to the discrete silicon dioxide nanoparticles of from 5:1 to 200:1.
